(19) United States
(12) Patent Application Publication
(10) Pub. No.: US 2015/0024025 A1
(43) Pub. Date: Jan. 22, 2015

(54) ALIGNED NANOFIBROUS STRUCTURES FOR AXONAL REGENERATION AFTER SPINAL CORD INJURY OR SURGERY

(71) Applicant: THE UAB RESEARCH FOUNDATION, Birmingham, AL (US)

(72) Inventor: Candace Floyd, Birmingham, AL (US)

(73) Assignee: The UABB Research Foundation, Birmingham, AL (US)

(21) Appl. No.: 14/383,475

(22) PCT Filed: Mar. 6, 2013
(86) PCT No.: PCT/US2013/029303

§ 371 Date: Sep. 5, 2014

Related U.S. Application Data
(60) Provisional application No. 61/607,390, filed on Mar. 6, 2012.

(51) Int. Cl. A61L 27/44; A61L 27/18; B29C 55/22; A61L 27/54

(52) U.S. Cl. CPC A61L 27/44; A61L 27/54; A61L 27/18; B29C 55/22; A61L 2430/32; A61L 2400/12; A61L 2300/602; A61L 2300/412; A61L 2300/414; B29K 2067/046
USPC 424/425; 514/17.7; 424/94.61; 424/94.1; 514/8.4; 514/8.3; 264/145

(57) ABSTRACT

Embodiments of the present disclosure provide for aligned nanofibrous polymer matrix structure, structures incorporating aligned nanofibrous polymer matrix structures, methods of using aligned nanofibrous polymer matrix structures, methods of making aligned nanofibrous polymer matrix structures, and the like.

### CLAIM OF PRIORITY TO RELATED APPLICATION

This application claims priority to co-pending U.S. provisional application entitled “FABRICATION OF AN ALIGNED 3D POLYMER SCAFFOLD TO PROMOTE AXONAL REGENERATION AND FUNCTIONAL RECOVERY AFTER SPINAL CORD INJURY OR SURGERY” having Ser. No. 61/607,390 filed on Mar. 6, 2012, which is entirely incorporated herein by reference.

### FEDERAL SPONSORSHIP

This invention was made with Government support under contract 5R01AR056665 awarded by the National Institutes of Health. The Government has certain rights in this invention.

### BACKGROUND

Spinal cord injury (SCI) induces devastating damage to neurons and their axons which results in permanent loss of function at and below the injury site. The initial mechanical impact to the spinal cord leads to a secondary injury cascade which involves multiple pathophysiological mechanisms and ultimately results in the formation of a glial-encapsulated cyst, which becomes a barrier to regeneration. This typically renders the persons who sustain such injury destined to a life of substantial disability. The human and economic cost of SCI is staggering. As of yet, there are no clinically-approved methods to repair the injured spinal cord to reduce the economic, physical and psychological toll associated with SCI.

### SUMMARY

Embodiments of the present disclosure provide for aligned nanofibrous polymer matrix structure, structures incorporating aligned nanofibrous polymer matrix structures, methods of using aligned nanofibrous polymer matrix structures, methods of making aligned nanofibrous polymer matrix structures, and the like.

In an exemplary embodiment, the structure includes a nanofiber hollow structure, wherein at least one type of aligned nanofibrous polymer matrix structure is disposed in the nanofiber hollow structure, each aligned nanofibrous polymer matrix structure having a distal end and a proximal end, wherein the aligned nanofibrous polymer matrix structure includes a plurality of aligned nanofibers, wherein the matrix structure encapsulates at least one component, wherein the component has a gradient-molecular orientation along the fiber-axis of the matrix structure, wherein molecular orientation is greater at the distal end of the aligned nanofibrous polymer matrix structure and decreases along the length of the aligned nanofibrous polymer matrix structure moving towards the proximal end, wherein the gradient-molecular orientation of the components modulates the release kinetics of the components.

In an exemplary embodiment, method of treating a spinal cord injury includes: disposing a nanofiber structure including aligned nanofibrous polymer matrix structures in a glial-encapsulated cyst of a patient, wherein the nanofiber structure has a distal end and a proximal end, wherein the nanofiber structure is positioned so that the distal end is positioned adjacent the distal nerve end of the spinal cord injury and the proximal end is positioned adjacent the proximal nerve end of the spinal cord injury, wherein each aligned nanofibrous polymer matrix structure has a distal end and a proximal end, wherein the aligned nanofibrous polymer matrix structure encapsulates at least one component, wherein the component has a gradient-molecular orientation along the fiber-axis of the aligned nanofibrous polymer matrix structure, wherein molecular orientation is greater at the distal end of the aligned nanofibrous polymer matrix structure and decreases along the length of the aligned nanofibrous polymer matrix structure moving towards the proximal end, wherein the gradient-molecular orientation of the components modulates the release kinetics of the components, wherein the release of the components from the proximal end of the aligned nanofibrous polymer matrix structure is greater than the release from the distal end of the aligned nanofibrous polymer matrix structure to produce a concentration gradient of the components, wherein the concentration gradient will promote directional axonal growth.

In an exemplary embodiment, a method of making an aligned nanofibrous polymer matrix structure includes: providing an aligned nanofibrous polymer matrix substrate, wherein the matrix substrate encapsulates at least one component; uni-axially drawing the aligned nanofibrous polymer matrix substrate until necking occurs in the aligned nanofibrous polymer matrix substrate to produce a gradient-molecular orientation of the component along the fiber-axis of the aligned nanofibrous polymer matrix substrate; and cutting a portion out of the aligned nanofibrous polymer matrix substrate to form an aligned nanofibrous polymer matrix structure, wherein the aligned nanofibrous polymer matrix structure has the gradient-molecular orientation of the component along the fiber-axis of the matrix structure.

In an exemplary embodiment, aligned nanofibrous polymer matrix structure includes: a plurality of aligned nanofibers, wherein nanofibers encapsulates at least one component, wherein the component has a gradient-molecular orientation along the fiber-axis of the matrix structure, wherein molecular orientation is greater at a distal end of the aligned nanofibrous polymer matrix structure and decreases along the length of the aligned nanofibrous polymer matrix structure moving towards a proximal end, wherein the gradient-molecular orientation of the components modulates the release kinetics of the components.

Other structures, methods, features, and advantages will be, or become, apparent to one with skill in the art upon examination of the following drawings and detailed description. It is intended that all such additional structures, systems, methods, features, and advantages be included within this description, be within the scope of the present disclosure, and be protected by the accompanying claims.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1(a) illustrates a cartoon of cystic cavity at epicenter of spinal cord lesion. FIG. 1(b) illustrates an image of a spinal cord after a contusion injury (bruising of the spinal cord) and shows that a cavity forms at the injury site with a small rim of spared tissue.

FIG. 2(a) illustrates preliminary data showing CLSM image of PCL/collagen 80/20 fibers. FIG. 2(b) illustrates AFM images of miscellaneously packed lamellae in single fiber with schematic drawing. FIG. 2(c) illustrates the uni-axial stretching will align the polymer molecules and give an orientation gradient toward the middle of “necking”.

FIG. 3 illustrates a molecular orientation gradient driving the delivery of neurotrophins in a timed manner to modulate regeneration using non-isotropic nanofibrous conduits for “long” gaps'. The aligned fibrous mats encapsulated with neurotrophin-3 (NT-3) or NGF will be packed in 3D tubular conduits after “uni-axially drawing until necking”.

FIG. 4 illustrates aligned nanofibers with encapsulated NGF. FIG. 4(a) illustrates preliminary data of SEM image of aligned fibers (˜300 nm diameter) of PLGA/collagen (80/20). FIG. 4(b) illustrates a fluorescent microscopic image showing the orientation of cultured cells along the fiber axis. FIG. 4(c) illustrates a digital photo of a 3D tubular conduit with uniform wall thickness. FIG. 4(d) illustrates a SEM cross sectional view of another 1 mm diameter conduit. FIG. 4(e) illustrates a CLSM image of NGF encapsulated nanoparticles incorporated electrospun fibers.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed. Terms defined in references that are incorporated by reference do not alter definitions of terms defined in the present disclosure or should such terms be used to define terms in the present disclosure they should only be used in a manner that is inconsistent with the present disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, polymer chemistry, molecular biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definition

As used herein, the terms “treatment”, “treating”, and “treat” are defined as acting upon a condition (e.g., spinal cord injury) to reduce or ameliorate the pharmacologic and/or physiologic effects of the condition. “Treatment,” as used herein, covers any treatment of a condition in a host (e.g., a mammal, typically a human or non-human animal of veterinary interest), and includes: impeding the further development of the condition and relieving the condition, i.e., causing regression of the condition and/or relieving one or more condition symptoms.

As used herein, the term “host,” “subject,” “patient,” or “organism” includes humans and mammals (e.g., mice, rats, pigs, cats, dogs, and horses). Typical hosts will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

The term “therapeutically effective amount” as used herein refers to that amount of a component being administered in a structure of the present disclosure that encourage nerve growth and/or decrease inhibiting agents.

### Discussion

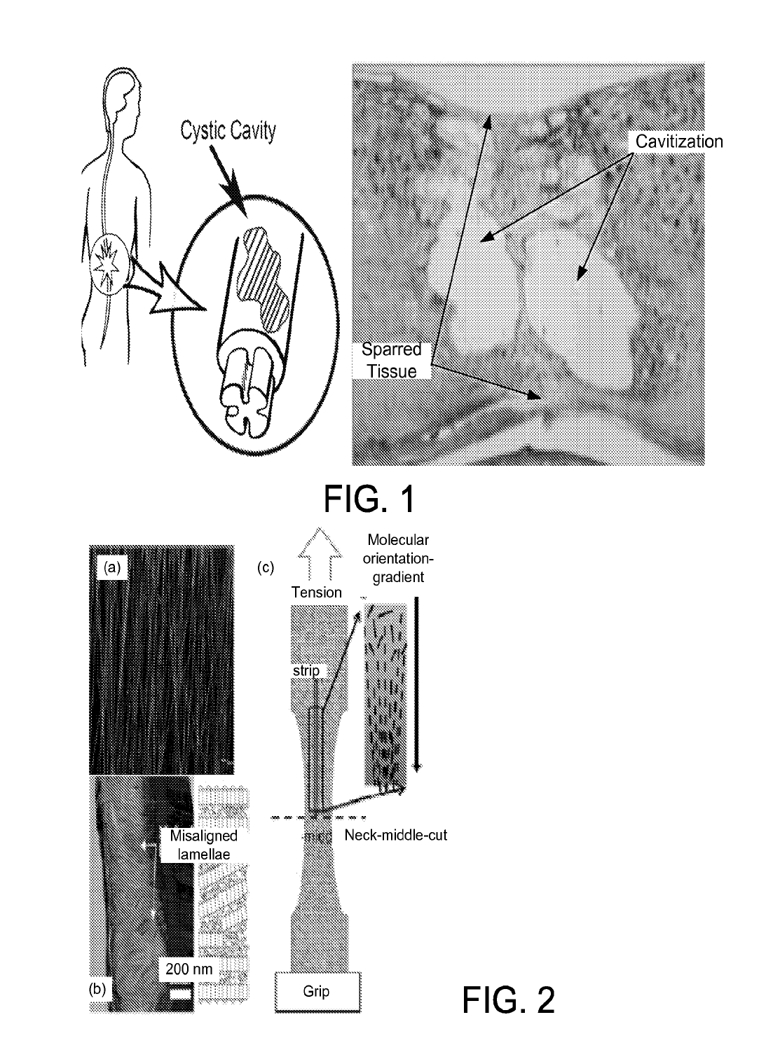

Embodiments of the present disclosure provide for aligned nanofibrous polymer matrix structures, structures incorporating aligned nanofibrous polymer matrix structures, methods of using aligned nanofibrous polymer matrix structures, methods of making aligned nanofibrous polymer matrix structures, and the like. In general, embodiments of the present disclosure can be used to directionally grow or encourage growth of nerve tissue, cells, vasculature, tissue, organ tissue, and the like, from one position to another position to promote functional recovery. Embodiment of the present disclosure can be used to promote axonal growth and regenerative response after a spinal cord injury or surgery. In particular, embodiments of the present disclosure can be used to directionally grow axon and regenerative response at a spinal cord injury site (e.g., glial-encapsulated cyst, See FIG. 1) from the proximate site of the injury to the distal site of the injury. In an embodiment, a structure can include growth factors and inhibition enzymes that can be released in a manner to facilitate directional growth of axons, where the structure provides a support for the axons, tissue, and cells to grow for a period of time and will degrade over time so that only the nerve tissue and cells remain. In an embodiment, the growth factors and/or inhibition enzymes can be released according to a gradient-molecular orientation that spatially controls the release, which acts to directionally guide the nerve tissue growth, or in other words, acts as a growth permissive nerve guidance system.

As mentioned above, an embodiment of the present disclosure includes an aligned nanofibrous polymer matrix structure, which includes a gradient-molecular orientation of one ore more components that can be used to directionally guide the nerve tissue growth. In an embodiment, the aligned nanofibrous polymer matrix structure includes a plurality of aligned nanofibers. In an embodiment, the nanofibers can be made of one or more polymers (e.g., polymer, co-polymer, block polymer, and the like) such as biopolymers that are biodegradable. In an embodiment, the polymer can be selected from the following: poly(lactide-co-glycolide) poly(lactide) (PLA), (PLGA), poly(caprolactone) (PCL), polylacide-co-caprolactone (PLCL), a polyhydroxy ester, collagen, gelatin, laminin, chitosan, silk, resilin, and a combination thereof. In an embodiment, the aligned nanofibrous polymer matrix structure can be formed using a high-voltage electrospinning process. In an embodiment, the nanofibrous polymer matrix layer can include other fibers such as microfibers. In an embodiment, nanofibrous polymer matrix layer can include other materials such as hydrogels.

In an embodiment, PGLA and be mixed with collagen in a ratio of about 100:10 to 10:100. In an embodiment, PCL and be mixed with chitosan in a ratio of about 100:10 to 10:100.

In an embodiment, the aligned nanofibrous polymer matrix structure can have a length of about 0.5 mm to 50 mm and a width of about 0.5 mm to 5 mm. In an embodiment, the aligned nanofibrous polymer matrix structure can have about 100 to 50000 nanofibers. In an embodiment, the nanofiber can have a length of about 0.5 mm to 50 mm and a diameter of about 100 nm to 2000 nm. In an embodiment, the aligned nanofibrous polymer matrix structure can include nanofibers having the same length and/or diameter or the nanofibers can have two or more lengths and/or diameters. In an embodiment, the nanofibers can be packed so as to have multiple layers of nanofibers (same or different types of nanofibers).

In an embodiment, the nanofibers can encapsulate (e.g., completely or partially) one or more components. The components can be encapsulated to control, at least partially, the release of the components. In an embodiment, the component can be a growth promoting factor, inhibiting agent, and the like. In an embodiment, each component is included in a therapeutically effective amount to achieve the desired result (e.g., improve growth, reduce growth inhibitors, and the like). In an embodiment, one or more components can be mixed with the polymers prior to forming the nanofiber and then the nanofiber can be formed so the components are encapsulated in the nanofiber.

In an embodiment, the growth promoting factor is included to encourage axonal outgrowth and regeneration on the aligned nanofibrous polymer matrix structure. In an embodiment, the growth promoting factor can be a glial cell-derived neutrophic factor, a nerve growth factor, a neurotrophin (e.g., neurotrophin-3, neurotrophin-4/5), ciliary-derived neurotrophic factor, nerve growth factor, brain-derived neurotrophic factor, and leukemia inhibitory factor. Nerve growth factor (NGF) and neurotrophin-3 (NT-3) are known to guide axons as well as promote axonal growth following injury to both the spinal cord and peripheral nerves. In an embodiment, the concentration of each of the growth promoting factor in the aligned nanofibrous polymer matrix structure can be about 10 microgram/ml to 1 milligram/ml. In particular, the concentration for neurotrophin can be about 10 microgram/ml to 1 milligram/ml.

In an embodiment, the inhibiting agent is included to reduce the number of inhibitory molecules present in the damaged tissue of the spinal cord. The main inhibitory molecules that substantially limit the regenerative capacity of the central nervous system (CNS) are known. These include inhibitors found in myelin such as Nogo-A, myelinassociated glycoprotein (MAG), oligodendrocyte myelin-associated glycoprotein (OMgp); and many types of chondroitin sulfate proteoglycans (CSPGs), which are secreted by astrocytes in the glial scar. Also inhibition can be induced by axon guidance molecules belonging to the semaphoring, ephrin and netrin families. Of these, PTEN/mTOR is of particular importance. Several of these inhibitors share common biochemical pathways and inhibition of upstream modulators is likely to inhibit several inhibitors. For example, inhibition of sialic acids or inhibitors of ganglioside biosynthesis (such as by the enzyme sialidase) would inhibit the function of MAG and likely other myelin inhibitors including NogoA and OMgp. Infusion of sialidase directly into the spinal cord injury lesion cavity for two weeks acutely post-injury induced functional recovery and limited axonal sprouting. Similarly, it is also well-established that the enzyme chondroitinase ABC breaks down several CSPGs and promotes axonal repair and regeneration in vivo after spinal cord injury. Taken together, inhibition of several key inhibitor molecules in and around the spinal cord injury lesion could be achieved by administration of enzymes to neutralize either the myelin-based inhibitory molecules or the astrocyte-based inhibitory molecules, or both. In an embodiment, the inhibiting agent can include inhibiting enzymes. In an embodiment, the inhibition agent can be a sialidase, chondrotinase ABC, Tarceva® (erlotinib hydrochloride, erlotinib (N-(3-ethynylphenyl)-6,7-bis(2-methoxyethoxy) quinazolin-4-amine)), and Nogo-66 antagonist peptide (NEP1-40). In an embodiment, the concentration of each of the inhibiting agents in the aligned nanofibrous polymer matrix structure can be about 2 U/ml to 100 U/mL.

In an embodiment, the aligned nanofibrous polymer matrix structure has a gradient-molecular orientation of the components along the fiber-axis of the aligned nanofibrous polymer matrix structure. In an embodiment, the molecular orientation is greater at one end (e.g., a distal end) of the aligned nanofibrous polymer matrix structure and decreases along the length of the aligned nanofibrous polymer matrix structure moving towards the other end (e.g., a proximal end). In an embodiment, the greater the molecular orientation, the more closely packed the components are within the aligned nanofibrous polymer matrix structure, which limits the ability of the components to be released from the aligned nanofibrous polymer matrix structure. Thus, as the molecular orientation decreases along the length of the aligned nanofibrous polymer matrix structure (from the distal end to the proximal end), the easier it is for the components to be released from the aligned nanofibrous polymer matrix structure. Consequently, the gradient-molecular orientation of the aligned nanofibrous polymer matrix structure acts as a way to spatially control the release of the components along the length of the gradient-molecular orientation, which is advantageous for the directional regeneration of the nerves in a spinal cord injury. In other words, the gradient-molecular orientation of the components modulates the release kinetics of the components in the aligned nanofibrous polymer matrix structure.

In an embodiment, the aligned nanofibrous polymer matrix structure can be made using the following method. First, an aligned nanofibrous polymer matrix substrate is uni-axially drawn (e.g., pulled) along the length of the nanofibers until necking (See FIG. 2) occurs in the aligned nanofibrous polymer matrix substrate to produce a gradient-molecular orientation of the component along the fiber-axis of the aligned nanofibrous polymer matrix substrate. The gradient-molecular orientation is greater at the central portion of the necked area and decreases along the length towards the non-necked area. The term “necking” refers to (the controlled stretching of the fibers uni-directionally under the influence of a force so that the middle-part of the specimen is narrowed or “necked” compared to the end. Necking causes the re-organization of the polymer chains with more aligned polymer molecules in the necked middle part as compared to a less aligned or random molecules present at the end (or non-necked portions). In an embodiment, the aligned nanofibrous polymer matrix substrate can be drawn using a dynamic mechanical analyzer at a low ramp force (e.g., about 0.001-0.1 N/min) under ambient conditions. Subsequently, a portion (e.g., a rectangle, a polygon, or other appropriate shape that can achieve the desired goal) of aligned nanofibrous polymer matrix substrate is cut (e.g., a blade, a laser, and the like) from the aligned nanofibrous polymer matrix substrate to form the aligned nanofibrous polymer matrix structure, where the aligned nanofibrous polymer matrix structure has the gradient-molecular orientation of the component along the fiber-axis of the matrix structure.

In an embodiment, the portion cut out is a rectangular portion from the aligned nanofibrous polymer matrix substrate. In an embodiment, the rectangular portion has a distal end and a proximal end, where the distal end starts in an area at about the middle of the aligned nanofibrous polymer matrix substrate that experienced necking and the proximal end starts in an area of the aligned nanofibrous polymer matrix substrate that did not experience necking. In an embodiment, the molecular orientation is greater at the distal end (necked area) of the aligned nanofibrous polymer matrix structure and decreases along the length of the aligned nanofibrous polymer matrix structure moving towards the proximal end (un-necked area).

In an embodiment, the aligned nanofibrous polymer matrix substrate includes a plurality of aligned nanofibers. In an embodiment, the nanofibers can be made of one or more polymers (e.g., polymer, co-polymer, block polymer, and the like) such as biopolymers that are biodegradable. In an embodiment, the polymer can be selected from the following: poly(lactide-co-glycolide) poly(lactide) (PLA), (PLGA), poly(caprolactone) (PCL), polylacide-co-caprolactone (PLCL), polydioxanone (PDO), polyhydroxy esters, collagen, gelatin, laminin, chitosan, silk, resilin and a combination thereof. In an embodiment, the components can be mixed with the polymers to form the nanofiber and then the nanofiber can be formed so the components are encapsulated in the nanofiber. In an embodiment where the structure includes nanofibers, the structure can be formed using a high-voltage electrospinning process.

In an embodiment, the aligned nanofibrous polymer matrix structure can have a length of about 0.5 mm to 50 mm and a width of about 0.5 mm to 5 mm. In an embodiment, the aligned nanofibrous polymer matrix structure can have about 100 to 50,000 nanofibers. In an embodiment, the nanofiber can have a length of about 0.5 mm to 50 mm and a diameter of about 100 nm to 2000 nm. In an embodiment, the aligned nanofibrous polymer matrix structure can include nanofibers having the same length and/or diameter or the nanofibers can have two or more lengths and/or diameters. In an embodiment, the nanofibers can encapsulate one or more components as described above.

In an embodiment, one or more aligned nanofibrous polymer matrix structures can be included in a structure such as a hollow structure to deliver the aligned nanofibrous polymer matrix structures to the spinal cord injury site (e.g., glial-encapsulated cyst). In an embodiment, the structure can be positioned in the glial-encapsulated cyst to properly orientate the aligned nanofibrous polymer matrix structure to facilitate the directional regeneration of the nerves from one side (proximal end) of the spinal cord injury to the other side (distal end) of the spinal cord injury. In other words, the positioning of the structure will encourage elongated axonal growth from the proximal side toward the distal side of the spinal cord injury.

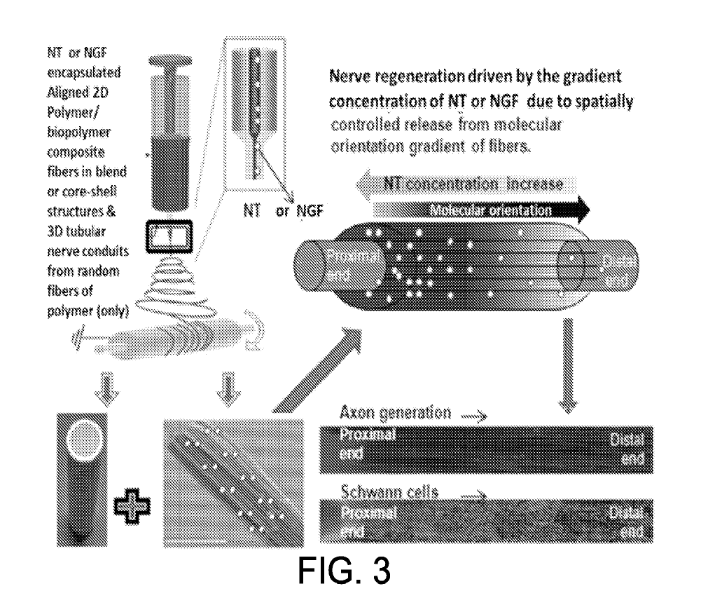

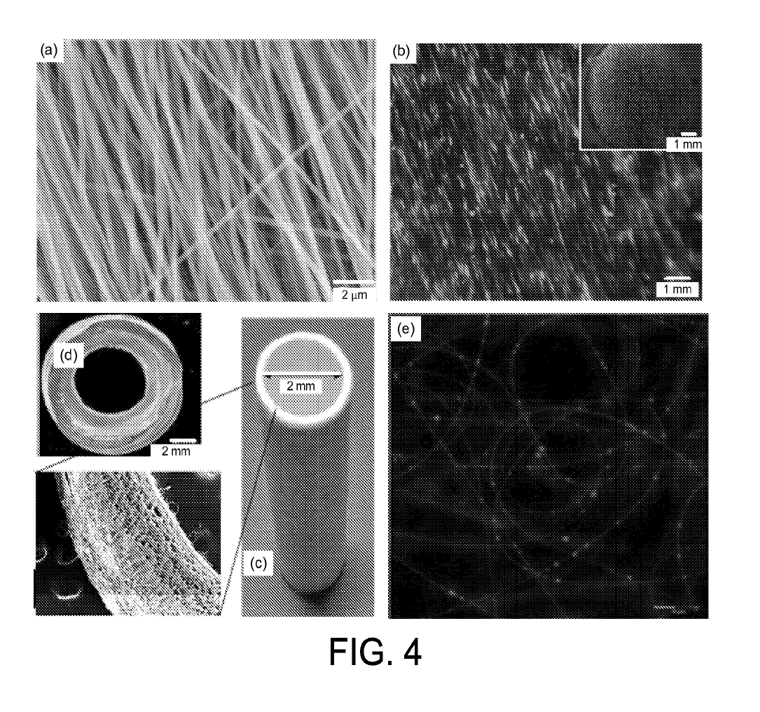

In an embodiment, the structure can be a nanofiber hollow structure, in particular, a nanofiber tube (See FIGS. 3 and 4). In an embodiment, at least one type of aligned nanofibrous polymer matrix structure is disposed in the nanofiber hollow structure. In an embodiment, each type of aligned nanofibrous polymer matrix structure can correspond to a aligned nanofibrous polymer matrix structure having a different component, a different combination of components, different concentration of a component, different concentration of a combination of components, a different gradient-molecular orientation, different type of nanofibers, and the like. Particular applications may include one or more types of aligned nanofibrous polymer matrix structures and those can be determined as needed to accomplish the specified goal. In an embodiment, the structure can include 1 to 25 aligned nanofibrous polymer matrix structures. In an embodiment, the number of aligned nanofibrous polymer matrix structures can be selected to adjust the therapeutically effective amount of one or more of the components.

In an embodiment, each aligned nanofibrous polymer matrix structure has a distal end and a proximal end. In an embodiment, each of the aligned nanofibrous polymer matrix structures is positioned so each distal end is located at the distal end of the nanofiber tube. The aligned nanofibrous polymer matrix structures are similarly oriented to facilitate the directional regeneration of the nerves. In an embodiment, it may be advantageous to have one or more aligned nanofibrous polymer matrix structures aligned differently (distal end of the aligned nanofibrous polymer matrix structure at the proximal end of the nanofiber hollow structure) if it is desired to have a component released at a different rate to achieve a desired result.

In an embodiment, the nanofibers of the structure can be made of a biopolymer such as poly(lactide-co-glycolide) (PLGA), poly(caprolactone) (PCL), polydioxanone (PDO), poly(ester urethane urea), and a combination thereof. In an embodiment, the nanofibers of the structure can encapsulate one or more components such as laminin, a laminin peptide, and a combination thereof. In an embodiment, the components can be mixed with the polymers to form the nanofiber and then the nanofiber can be formed so the components are encapsulated in the nanofiber. In an embodiment where the structure includes nanofibers, the structure can be formed using a high-voltage electrospinning process. In an embodiment, the concentration of the laminin in the nanofiber structure can vary as needed depending on the use and desired result to be achieved.

In an embodiment, the nanofibers of the structure can be aligned or not aligned. In an embodiment, the nanofibers of the structure can have a length of about 0.5 mm to 50 mm and a diameter of about 100 nm to 2000 nm.

In an embodiment, the structure has dimensions so that it can be disposed in the spinal cord injury site. In an embodiment, the structure has a hollow area to include one or more aligned nanofibrous polymer matrix structures. In an embodiment, the structure can have a length of about 0.5 mm to 50 mm and a width of about 0.5 mm to 5 mm. In an embodiment, the structure has a hollow area to include one or more aligned nanofibrous polymer matrix structures. In an embodiment where the structure is a nanofiber tube, the nanofiber tube can have a length of about 0.5 mm to 50 mm, an inner diameter of about 1 mm to 5 mm, and an outer diameter of about 1.2 mm to 6 mm. In an embodiment, the aligned nanofibrous polymer matrix structure can include 1 to 25 aligned nanofibrous polymer matrix structures.

In an embodiment, a spinal cord injury can be treated using embodiments of the present disclosure. In an embodiment, a structure, such as a nanofiber structure, including one or more types of aligned nanofibrous polymer matrix structures can be positioned in a glial-encapsulated cyst of a patient. In an embodiment, the nanofiber structure has a distal end and a proximal end, where the nanofiber structure is positioned so that the distal end is positioned adjacent the distal nerve end of the spinal cord injury and the proximal end is positioned adjacent the proximal nerve end of the spinal cord injury. In an embodiment, the distal end of each aligned nanofibrous polymer matrix structure is oriented with the distal end nanofiber structure and the proximal end of each aligned nanofibrous polymer matrix structure is oriented with the proximal end of the nanofiber structure. As a result, the distal end of each of the aligned nanofibrous polymer matrix structures is positioned at the distal nerve end of the spinal cord injury (See FIG. 3). In an embodiment, the release of the components from the proximal end of the aligned nanofibrous polymer matrix structure is greater than the release from the distal end of the aligned nanofibrous polymer matrix structure to produce a concentration gradient of the components, where the concentration gradient will promote directional axonal growth and regeneration of nerve tissue from the proximate side to the distal side of the spinal cord injury.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to the measurement technique and the type of numerical value. In addition, the phrase “about ‘x’ to ‘y’” includes “about to about ‘y’”.

Many variations and modifications may be made to the above-described embodiments. All such modifications and variations are intended to be included herein within the scope of this disclosure and protected by the following claims.

At least the following is claimed:

1. A structure, comprising:
a nanofiber hollow structure, wherein at least one type of aligned nanofibrous polymer matrix structure is disposed in the nanofiber hollow structure, each aligned nanofibrous polymer matrix structure having a distal end and a proximal end, wherein the aligned nanofibrous polymer matrix structure includes a plurality of aligned nanofibers, wherein the matrix structure encapsulates at least one component, wherein the component has a gradient-molecular orientation along the fiber-axis of the matrix structure, wherein the molecular orientation is greater at the distal end of the aligned nanofibrous polymer matrix structure and decreases along the length of the aligned nanofibrous polymer matrix structure moving towards the proximal end, wherein the gradient-molecular orientation of the components modulates the release kinetics of the components.

2. The structure of claim 1, wherein the nanofiber hollow structure is made of a polymer selected from the group consisting of: poly(lactide-co-glycolide) (PLGA), poly(caprolactone) (PCL), polydioxanone (PDO), poly(ester urethane urea), and a combination thereof; and wherein the nanofibers of the aligned nanofibrous polymer matrix structure are made of a polymer selected from the group consisting of: poly(lactide-co-glycolide) poly(lactide) (PLA), (PLGA), poly(caprolactone) (PCL), polylacide-co-caprolactone (PLCL), a polyhydroxy esters, collagen, gelatin, laminin, chitosan, silk, resilin, and a combination thereof.

3. (canceled)

4. The structure of claim 1, wherein at least one component is selected from a growth promoting factor or an inhibition agent.

5. (canceled)

6. The structure of claim 1, wherein at least one component is a growth promoting factor and at least one component is an inhibition agent.

7. The structure of claim 6, wherein at least one growth promoting factor is selected from a glial cell-derived neutrophic factor, a nerve growth factor, a neurotrophin, ciliary-derived neurotrophic factor, nerve growth factor, brain-derived neurotrophic factor, and leukemia inhibitory factor and wherein at least one inhibition agent is selected from: a sialidase, chondrotinase ABC, erlotinib, and Nogo-66 antagonist peptide (NEP1-40).

8. (canceled)

9. The structure of claim 1, wherein the nanofiber hollow structure has a length of about 0.5 mm to 50 mm, an inner diameter of about 0.5 mm to 5 mm, and an outer diameter of about 1.2 mm to 6 mm; wherein the aligned nanofibrous polymer matrix structure has a length of about 0.5 mm to 50 mm, and a width of about 0.5 mm to 5 mm.

10. (canceled)

11. The structure of claim 1, wherein a first aligned nanofibrous polymer matrix structure includes a growth promoting factor, wherein a second aligned nanofibrous polymer matrix structure includes an inhibition agent.

12. The structure of claim 1, wherein a first aligned nanofibrous polymer matrix structure includes a growth promoting factor and an inhibition agent.

13. (canceled)

14. (canceled)

15. (canceled)

16. The structure of claim 1, wherein the nanofibers of the nanofiber hollow structure encapsulate at least one component that is selected from laminin, a laminin peptide, and a combination thereof.

17. (canceled)

18. A method of treating a spinal cord injury, comprising:
disposing a nanofiber structure including aligned nanofibrous polymer matrix structures in a glial-encapsulated cyst of a patient, wherein the nanofiber structure has a distal end and a proximal end, wherein the nanofiber structure is positioned so that the distal end is positioned adjacent the distal nerve end of the spinal cord injury and the proximal end is positioned adjacent the proximal nerve end of the spinal cord injury, wherein each aligned nanofibrous polymer matrix structure has a distal end and a proximal end, wherein the aligned nanofibrous polymer matrix structure encapsulates at least one component, wherein the component has a gradient-molecular orientation along the fiber-axis of the aligned nanofibrous polymer matrix structure, wherein molecular orientation is greater at the distal end of the aligned nanofibrous polymer matrix structure and decreases along the length of the aligned nanofibrous polymer matrix structure moving towards the proximal end, wherein the gradient-molecular orientation of the components modulates the release kinetics of the components, wherein the release of the components from the proximal end of the aligned nanofibrous polymer matrix structure is greater than the release from the distal end of the aligned nanofibrous polymer matrix structure to produce a concentration gradient of the components, wherein the concentration gradient will promote directional axonal growth.

19. A method of making an aligned nanofibrous polymer matrix structure, comprising,
providing an aligned nanofibrous polymer matrix substrate, wherein the matrix substrate encapsulates at least one component;
uni-axially drawing the aligned nanofibrous polymer matrix substrate until necking occurs in the aligned nanofibrous polymer matrix substrate to produce a gradient-molecular orientation of the component along the fiber-axis of the aligned nanofibrous polymer matrix substrate; and
cutting a portion out of the aligned nanofibrous polymer matrix substrate to form an aligned nanofibrous polymer matrix structure, wherein the aligned nanofibrous polymer matrix structure has the gradient-molecular orientation of the component along the fiber-axis of the matrix structure.

20. The method of claim 19, wherein cutting includes cutting a rectangular portion from the aligned nanofibrous polymer matrix substrate, wherein the rectangular portion has a distal end and a proximal end, wherein the distal end starts in an area at about the middle of the aligned nanofibrous polymer matrix substrate that experienced necking and the proximal end starts in an area of the aligned nanofibrous polymer matrix substrate that did not experience necking so that the aligned nanofibrous polymer matrix structure has the gradient-molecular orientation of the component along the fiber-axis of the aligned nanofibrous polymer matrix substrate, wherein the molecular orientation is greater at the distal end of the aligned nanofibrous polymer matrix structure and decreases along the length of the aligned nanofibrous polymer matrix structure moving towards the proximal end.

21. The structure of claim 1, where the aligned nanofibrous polymer matrix structure include:
a plurality of aligned nanofibers, wherein nanofibers encapsulates at least one component, wherein the component has a gradient-molecular orientation along the fiber-axis of the matrix structure, wherein molecular orientation is greater at a distal end of the aligned nanofibrous polymer matrix structure and decreases along the length of the aligned nanofibrous polymer matrix structure moving towards a proximal end, wherein the gradient-molecular orientation of the components modulates the release kinetics of the components.

22. The structure of claim 21, wherein the nanofibers are made of a polymer selected from the group consisting of: poly(lactide-co-glycolide) poly(lactide) (PLA), (PLGA), poly(caprolactone) (PCL), polylacide-co-caprolactone (PLCL), a polyhydroxy ester, collagen, gelatin, laminin, chitosan, silk, resilin, and a combination thereof.

23. (canceled)

24. (canceled)

25. The structure of claim 21, wherein at least one component is a growth promoting factor and at least one component is an inhibition agent; wherein at least one growth promoting factor is selected from a glial cell-derived neutrophic factor, a nerve growth factor, a neurotrophin, ciliary-derived neurotrophic factor, nerve growth factor, brain-derived neurotrophic factor, and leukemia inhibitory factor; and wherein at least one inhibition agent is selected from: a sialidase, chondrotinase ABC, erlotinib, and Nogo-66 antagonist peptide (NEP1-40).

26. (canceled)

27. (canceled)

28. (canceled)
